(19) United States
(12) Patent Application Publication
(10) Pub. No.: US 2026/0306876 A1
(43) Pub. Date: Oct. 8, 2026

(54) SYSTEMS AND METHODS FOR STEREOTACTIC GUIDED RADIATION THERAPY USING COMPUTER VISION SYSTEMS FOR PATIENT SPECIFIC BODY MODELS

(71) Applicant: The Regents of the University of Colorado, a body corporate, Denver, CO (US)

(72) Inventors: David Thomas, Bala Cynwyd, PA (US); Danna Gurari, Boulder, CO (US)

(21) Appl. No.: 19/483,838

(22) PCT Filed: May 10, 2024
(86) PCT No.: PCT/US2024/028777

§ 371 Date: Nov. 12, 2025

Related U.S. Application Data
(60) Provisional application No. 63/501,770, filed on May 12, 2023.

(51) Int. Cl. A61N 5/10

(52) U.S. Cl. CPC A61N 5/1049; A61N 2005/1051; A61N 2005/1061; A61N 2005/1074

(57) ABSTRACT

A computer-implemented method and system can generate a dynamic anatomical representation of an individual. A system can obtain demographic data of the individual and a series of indexed three-dimensional (3D) medical imaging datasets corresponding to distinct phases of a respiratory cycle. A surface mesh model can be created to represent an external body contour. A skeletal mesh model can be derived using the surface mesh model, demographic data, and a comparative anatomical data library. Real-time image data depicting external surface features can be captured, and a dynamic anatomical representation can be generated by registering these images with the skeletal mesh model. The representation can adapt to reflect changes in a pose of the individual.

### FIELD

The present disclosure generally relates to the field of computer vision and, more particularly, to generating dynamic anatomical representations using medical imaging data.

### BACKGROUND

Radiation therapy (RT) can be an important component of cancer treatment protocols, administered to over half a million patients each year in the United States alone. This therapy aims to deliver ionizing radiation doses to malignant tumors to eradicate or control cancerous growths. While radiation therapy can be highly effective, ensuring the precise delivery of radiation to the tumor while minimizing exposure to surrounding healthy tissues remains a significant challenge.

One of the primary complexities in radiation therapy arises from the need to accurately target tumors, which can shift position within the body due to patient movements, including respiration. For example, abdominal tumors may shift significantly due to breathing, complicating the precise delivery of radiation. Misalignment, even by small margins, can lead to irradiation of healthy tissues, resulting in severe side effects such as difficulty swallowing, nausea, radiation fibrosis, and other organ-specific adverse effects.

To address these challenges, various imaging techniques are employed to facilitate accurate tumor localization and treatment planning. Computed Tomography (CT) imaging is commonly used due to its ability to provide detailed images of both the tumor and the surrounding anatomy. CT scans are typically taken on treatment dates to create a reference for radiation delivery. However, the ionizing nature of CT imaging contributes to the total radiation dose received by the patient, posing additional risks.

Stereotactic Body Radiation Therapy (SBRT) represents an advancement in radiation delivery, offering the potential to deliver high doses of radiation with precision over a fewer number of treatments. SBRT has proven particularly effective for treating tumors previously considered refractory to radiation, such as those in the liver or pancreas. Despite its benefits, SBRT requires extremely precise targeting due to the high radiation doses involved, which can be complicated by the internal motion of tumors due to natural movements of the body, such as breathing.

Surface Guided Radiation Therapy (SGRT) is another technique that has been developed to improve the setup and delivery of radiation therapy. SGRT utilizes external imaging to monitor and track the position of the patient's skin in real-time, aiding in the correct positioning of the patient relative to the radiation beam. This method helps in aligning radiation delivery based on external body movements. However, the accuracy of SGRT can be limited when dealing with internal organ movement that is not directly correlated with surface movements, particularly for tumors located deep within the body or in areas prone to significant internal motion. Furthermore, SGRT can encounter limitations due to its simplistic registration approach (STMA), which may not fully account for the diversity in patient body types, such as variations between thin and obese patients.

### SUMMARY

Some embodiments of the present disclosure disclose a method for generating a dynamic anatomical representation of an individual, suitable for assisting in guiding adaptive procedures. The method can include obtaining demographic data of an individual. The demographic data can include an indication of at least one of gender, race, or body mass index (BMI). The method can include obtaining a series of indexed three-dimensional (3D) medical imaging datasets of the individual. Each dataset can correspond to a distinct phase of a respiratory cycle of the individual and depicting internal anatomical structures of the individual. The method can include generating a surface mesh model for the individual based on the series of indexed 3D imaging datasets. The surface mesh model can represent an external contour of a body of the individual. The method can include deriving a skeletal mesh model based on the surface mesh model, the demographic data, and a comparative anatomical data library. The comparative anatomical data library can include a set of anatomical templates. Deriving the skeletal mesh model can include using the demographic data to select an anatomical template that satisfies predefined thresholds for anatomical congruence with characteristics of the individual as represented by the demographic data, and correlating external surface features from the surface mesh model with internal skeletal configurations of the selected anatomical template. The method can include obtaining real-time image data depicting external surface features of the individual; and generating a dynamic anatomical representation by registering the real-time image data with the skeletal mesh model. The dynamic anatomical representation can adapt to reflect changes in a pose of the individual.

The method of the previous paragraph can include any one or more of following steps or features. The series of indexed three-dimensional (3D) computed tomography (CT) imaging datasets can include at least two datasets, one corresponding to a full inhalation phase and one corresponding to a full exhalation phase of a respiratory cycle of the individual. Each of the series of indexed 3D imaging datasets can be a CT dataset. The series of indexed 3D CT datasets can collectively form a four-dimensional (4D) CT dataset, capturing dynamic anatomical changes over time due to the respiratory cycle of the individual.

The method of any of the previous paragraphs can include any one or more of following steps or features. The method can include updating the surface mesh model and the skeletal mesh model continuously using a computer vision system to reflect real-time movements of the individual. Updates can include adjustments based on detected breathing cycles of the individual. Obtaining real-time image data can include capturing images of fiducial markers placed on an external surface of the individual. The captured images can be used to track movement due to respiratory and other body movements. Motion of skin of the individual, as indicated by the movement of the fiducial markers, can be utilized to estimate related movements of an anatomy of the individual. The dynamic anatomical representation can include a predictive model of skeletal movement derived from the skeletal mesh model and the real-time image data. The predictive model can account for variations in skeletal position due to respiratory movements.

The method of any of the previous paragraphs can include any one or more of following steps or features. The demographic data can be used to refine the anatomical template selection by comparing a height, weight, and/or gender of the individual with templates from the comparative anatomical data library to find a closest match. Radiation therapy can be applied using the dynamic anatomical representation to control dosage and/or timing of radiation. The method can include obtaining target region data. The target region data can include an indication of a location, size, and/or shape of a target region. Generating the dynamic anatomical representation can include incorporating the target region data into the skeletal mesh model to provide a target region-specific representation.

The method of any of the previous paragraphs can include any one or more of following steps or features. The dynamic anatomical representation can be configured to track changes in target region location relative to the skeletal mesh model across distinct respiratory phases to provide adaptive guidance. The method can include predicting potential movements of the target region based on respiratory phases of the individual. The skeletal mesh model can be updated to reflect anticipated target region shifts during the adaptive guidance. The method can include delivering radiation therapy by activating a therapeutic beam when the target region aligns within a predetermined focal area, as indicated by the dynamic anatomical representation. The dynamic anatomical representation can be intended for planning or simulation purposes, to inform healthcare professionals in developing or refining a radiation therapy plan.

Some embodiments of the present disclosure disclose a system for generating a dynamic anatomical representation of an individual. The system can include a processor circuit, operatively coupled to a computer vision system. The processor circuit can be configured to obtain a series of indexed three-dimensional (3D) medical imaging datasets of the individual. Each dataset can correspond to a distinct phase of a respiratory cycle of the individual and depicting internal anatomical structures of the individual. The processor circuit can be configured to generate a surface mesh model for the individual based on the series of indexed 3D imaging datasets. The surface mesh model can represent an external contour of a body of the individual. The processor circuit can be configured to derive a skeletal mesh model based on the surface mesh model, the demographic data, and a comparative anatomical data library. The comparative anatomical data library can include a set of anatomical templates. Deriving the skeletal mesh model can include using the demographic data to select an anatomical template that satisfies predefined thresholds for anatomical congruence with characteristics of the individual as represented by the demographic data, and correlating external surface features from the surface mesh model with internal skeletal configurations of the selected anatomical template. The processor circuit can be configured to obtain real-time image data depicting external surface features of the individual; and generating a dynamic anatomical representation by registering the real-time image data with the skeletal mesh model. The dynamic anatomical representation can adapt to reflect changes in a pose of the individual.

The system of any of the previous paragraphs can include any one or more of following steps or features. The series of indexed three-dimensional (3D) computed tomography (CT) imaging datasets can include at least two datasets, one corresponding to a full inhalation phase and one corresponding to a full exhalation phase of a respiratory cycle of the individual. The processor circuit can be configured to obtain tumor data of the individual. The tumor data can include an indication of the location, size, and/or shape of a tumor. The processor circuit can generate the dynamic anatomical representation by incorporating the tumor data into the skeletal mesh model to provide a tumor-specific representation.

Some embodiments of the present disclosure disclose a computer-implemented method for adapting radiation therapy based on positioning of a target region. The method can include obtaining a series of three-dimensional (3D) medical imaging datasets depicting an individual and a target region at different phases of a respiratory cycle of the individual; generating a dynamic anatomical model of the individual based on the 3D imaging datasets, wherein the dynamic anatomical model can include a representation of the target region, wherein generating can include: constructing a surface mesh model representing an external contour of a body of the individual, deriving an internal skeletal model from the surface mesh model by correlating external surface features with internal skeletal configurations using a comparative anatomical data library, and registering the internal skeletal model with the representation of the target region from the 3D imaging datasets to create the dynamic anatomical model; based on the dynamic anatomical model, determining an alignment of the target region with a predetermined focal area of a therapeutic beam during different phases of the respiratory cycle; and controlling delivery of radiation therapy based on the alignment, wherein radiation can be activated when the target region can be within the predetermined focal area and deactivated when the target region moves out of the predetermined focal area.

The method of any of the previous paragraphs can include any one or more of following steps or features. Determining the alignment of the target region with the predetermined focal area can be intended for planning or simulation purposes to inform the development or refinement of a radiation therapy plan.

### BRIEF DESCRIPTION OF THE DRAWINGS

FIG. 1A illustrates an example partial body CT scan of a patient, in accordance with some embodiments of the inventive concepts.

FIG. 1B illustrates an example transformation of the partial body CT scan into a full body surface mesh, in accordance with some embodiments of the inventive concepts.

FIG. 1C illustrates an example inference of a skeleton mesh from the full body surface mesh, in accordance with some embodiments of the inventive concepts.

FIG. 1D illustrates an example ground truth skeleton mesh extracted directly from the CT scan, in accordance with some embodiments of the inventive concepts.

FIG. 2A illustrates example performance of an example Avatar Guided Radiation Therapy (AGRT) system, showing the Root Mean Square Error (RMSE) across various anatomical bone groups.

FIG. 2B shows a density plot of the mean RMSE values for overall skeletal accuracy, categorized by patient demographics, to demonstrate example adaptability and effectiveness of the AGRT system across different patient conditions.

FIGS. 2C-2G illustrates a comparison of an example AGRT solution and the status quo in clinical settings of STMA with respect to (2C) treatment-relevant bone groups, (2D, 2E) overall skeletons, and (2F, 2G) influence of body mass index.

FIG. 3 is a block diagram illustrating an SGRT system including a computer vision system configured to capture the pose of a patient in the SGRT environment to generate a patient specific skin mesh model and including a real-time therapy guidance controller to compensate for the movement of the patient's anatomy based on the patient's breathing in some embodiments according to the present inventive concept.

FIG. 4 is a schematic illustration of a patient in a particular pose on a platform in the SGRT environment in some embodiments according to the present inventive concept.

FIG. 5 is a schematic illustration of three exemplary patients in respective poses on the platform captured by the computer vision system and the associated avatar selected for each patient in the pose based on a plurality of patient characteristics wherein the patient avatar is modified by the patient pose to generate the patient specific skin mesh model in some embodiments according to the present inventive concept.

FIG. 6 is a block diagram of processing operations configured to generate a skeletal model based on the patient specific skin mesh model wherein the skeletal model based is modified using patient specific CT data to align the skeletal model to the patient skeleton to provide a patient specific skeletal position model in some embodiments according to the present inventive concept.

FIG. 7 is an illustration of selected avatar in a particular pose on a platform in the SGRT environment, the selected avatar is selected based on a body type of the patient in some embodiments according to the present inventive concept.

FIG. 8 is an illustration of the selected patient avatar in FIG. 7 having been modified based on patient pose, gender, race, and BMI to provide a patient avatar annotated with a patient specific skin mesh model thereon in some embodiments according to the present inventive concept.

FIG. 9 is an illustration of the patient avatar in FIG. 8 annotated with a patient specific skeletal position model generated based on the patient specific skin mesh model as shown in FIG. 8 and FIG. 3 in some embodiments according to the present inventive concept.

FIG. 10 is an illustration of a specific patient annotated to reflect the measured patient skin movement locations and amounts due to breathing which can be used to determine anatomical movement in some embodiments according to the present inventive concept.

FIG. 11 is a flow diagram illustrative of an embodiment of a routine 1100 for generating a dynamic anatomical representation of an individual, suitable for assisting in guiding adaptive procedures.

### DETAILED DESCRIPTION

In traditional radiation therapy, the radiation equipment is generally static in that it does not follow the tumor's movements. Instead, the radiation is administered based on precise timing, activated when the tumor aligns within the predetermined focal area of the therapeutic beam, and deactivated when it moves out of range. This approach relies heavily on accurate tumor localization. Despite the advancements in imaging and tracking technologies, challenges remain in effectively adapting radiation delivery with dynamic anatomical changes, such as those caused by natural movements like breathing. Consequently, this can lead to misalignment between the radiation target and the tumor, resulting in insufficient radiation dose delivery to the tumor and/or unnecessary irradiation of healthy tissues. Such misalignment increases the risk of adverse side effects and reduces the overall efficacy of the treatment.

Some embodiments according to the present inventive concept address these or other challenges by utilizing a computer vision system to generate dynamic, patient-specific anatomical models that can more accurately reflect the patient's current anatomical state, including any movements or changes in positioning. These models can be used to guide the delivery of radiation, increasing the likelihood that the radiation beam is activated when the tumor aligns with the focal area of the therapeutic beam, thus increasing the likelihood that the radiation dose is accurately focused on the tumor, regardless of its movement within the body, and reducing the likelihood of irradiation of healthy tissues.

Some embodiments according to the present inventive concept leverage computer vision techniques to continuously capture and update a model of the patient's external anatomy. This model can be integrated or registered with internal imaging data, such as CT scans, to create a dynamic representation of the patient's anatomy. In some cases, this dynamic representation can include surface and skeletal structures.

In some embodiments, systems or methods in accordance with some aspects of the present inventive concepts can dynamically update a patient-specific model to reflect changes due to breathing or other movements. Such a real-time adaptation can improve tumor localization and treatment planning. For example, this enhancement can allow the radiation beam to be precisely controlled—turned on when the tumor is within the effective treatment zone and turned off when it moves out of the effective treatment zone. As a result, this approach can significantly reduce the irradiation of healthy tissues and/or can increase a likelihood that the radiation dose administered is more accurately focused on the tumor, enhancing both the safety and effectiveness of cancer treatment.

In some embodiments, systems or methods in accordance with some aspects of the present inventive concepts can seamlessly integrate with existing radiation therapy systems. Such an integration can advantageously allow for more precise radiation dose control, reducing exposure to healthy tissues and increasing the therapeutic impact on the tumor.

Some embodiments according to the present inventive concept can provide for the radiation therapy that more accurately aligns radiation with the target tumors. For example, some embodiments according to the present inventive concepts can leverage artificial intelligence, based on a novel computer vision technique, to create a personalized breathing ‘avatar’ of patients receiving radiation to allow more precise radiation delivery specifically configured and controlled for each patient.

Some embodiments according to the present inventive concept can provide an improved patient positioning system using a novel patient-specific skin mesh model that can combine a skeletal-pose machine-learning model with a computer vision system to enable marker-less and non-invasive real-time 3D human anatomy tracking. In still further embodiments according to the present inventive concept, a computer vision system can be used to determine a patient's pose in the therapy environment that can be used to generate a skeletal-pose computer vision model that correlates an external (skin mesh of the patient) to an internal (biomechanical) anatomy for the patient, thereby aligning the patient-specific model to a patient's CT imaging. In still further embodiments according to the present inventive concept, the patient specific skin mesh model can be used with a patient-specific breathing motion model acquired from individual patient 4D-CTs to control delivery of the radiation during therapy by compensating for the motion of the tumors due to patient breathing. Further, these systems can also provide a process for artificial intelligence (AI)/computer vision assisted alignment during Stereotactic Body Radiation Therapy (SBRT).

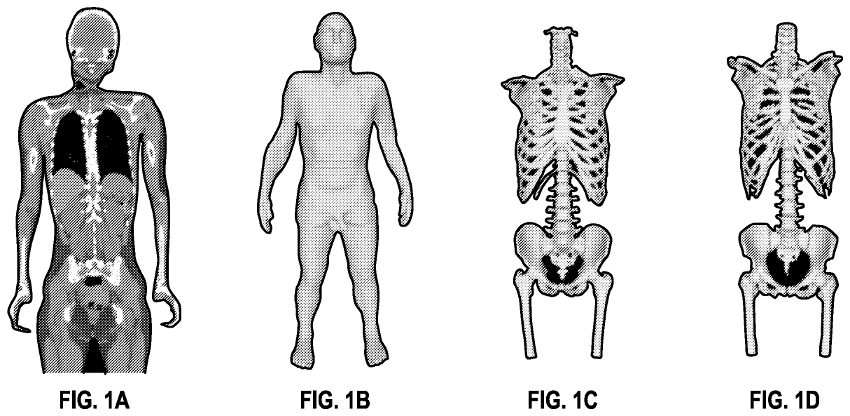

Some embodiments according to the present inventive concept include an Avatar Guided Radiation Therapy (AGRT) system. In some cases, the AGRT system can use a personalized patient avatar include both a body surface model and an internal skeletal model. In some cases, the AGRT system does not rely only on an unstable 3D body surface model of the patient, which can vary over time for many reasons (e.g., weight changes, breathing motion), and instead relies on a more stable skeletal model that stays relatively static over time. In some cases, the AGRT system adapts state-of-the-art computer vision methods in order to convert an initial CT scan into a 3D body surface mesh and then into a skeleton mesh, as exemplified in FIGS. 1A-1D.

FIG. 1A illustrates an example partial body CT scan of a patient, FIG. 1B illustrates an example transformation of the partial body CT scan into a full body surface mesh, FIG. 1C illustrates an example inference of a skeleton mesh from the full body surface mesh, and FIG. 1D illustrates an example ground truth skeleton mesh extracted directly from the CT scan. Evaluation on a retrospective clinical database of 198 CT scans from 49 patients shows an example AGRT system yields greater robustness (e.g., fewer large errors), greater consistency over time (e.g., consistently fewer large errors over the duration of the treatment plan), and greater equity (e.g., for obese patients) compared to the current state-of-the-art, SGRT.

Full-body surface models are utilized across various fields, encompassing computer graphics, computer vision, and virtual reality, for a myriad of applications. Recent advancements have predominantly involved the utilization of large datasets of 3D human body scans for model learning. Some embodiments according to the present inventive concept demonstrate an improvement to the prevailing state-of-the-art Skinned Multi Person Linear Model (SMPL) model, following training on 1,786 high-resolution 3D scans capturing individuals in diverse poses, to efficiently and expediently capture the shape and posture of patients undergoing cancer treatment.

Skeleton models may be introduced in contexts such as computer graphics and computer vision, notably for tasks like action recognition. However, the requisites within these domains often entail the modeling of select individual bones or bone groups. In medical applications, where precision is paramount due to the implications for human lives, a more intricate representation of skeletal anatomy becomes imperative. Consequently, some embodiments according to the present inventive concept adopt the Obtaining Skeleton Shape from Outside (OSSO) model, which produces a highly realistic skeletal anatomy of the human body based on the 3D full-body surface model.

Some embodiments according to the present inventive concept relate to personalized anatomic models, derived from both external and internal observations. In some cases, this disclosure represents a novel use of personalized anatomic models in the context of radiation therapy for cancer patients.

Disclosed herein is an Avatar Guided Radiation Therapy (AGRT) system. In some cases, the AGRT system uses skeleton-based modeling in addition to body surface modeling to guide radiation treatment for cancer patients. Evaluation on a retrospective clinical database shows that an example AGRT system achieves greater robustness (e.g., fewer large errors), greater consistency over time (e.g., consistently fewer large errors over the duration of the treatment plan), and greater equity (e.g., for obese patients) compared to the current state-of-the-art in clinical settings, Surface Guided Radiation Therapy (SGRT). In some embodiments, the disclosed AGRT system can achieve less than 2 mm accuracy and can be used independently for image-guided radiation therapy.

### Personalized Skeleton Models

Disclosed herein is an example workflow for converting an input CT scan from a patient into a personalized skeleton model.

For each patient, multiple types of information can be utilized. For example, the inputs can include, but are not limited to, a patient's height (e.g., in meters), weight (e.g., in lbs), gender, or age. In some cases, one or more internal images (e.g., CT scans) of the patient are also utilized. In some cases, the CT scans can range from full-body scans, where the entire body of the patient from skull to toes is observed, to partial-body scans, which may only show regions that are relevant or proximate to an area for RT.

In some cases, a CT scan is a 3D volume, represented as a stack of 2D grayscale images that were captured by sending a series of X-ray beams at the patient's body from different angles. Each pixel value in the image slices can reflect a Hounsfield unit (HU), which is a physically-derived value specifying the radio-density of tissue relative to distilled water (at standard temperature and pressure). HU values are a linear transformation of the attenuation coefficients of the X-ray beam (see Equation 1) reflecting that tissue's density is proportional to the attenuation of an X-ray beam. Distilled water (at standard temperature and pressure) is arbitrarily defined to be zero HU and air defined as −1000 HU. More dense tissue, with greater X-ray beam absorption, have positive values whereas less dense tissue with less X-ray beam absorption have negative values. The upper limits for HU values can reach 1000 for bones, 2000 for dense bones like the cochlea, and more than 3000 for metals like steel or silver.

$$H {U}_{material} = \frac{{μ}_{material} - {μ}_{water}}{{μ}_{water}} \times 1000 \qquad ( Equation 1 )$$

In some cases, the content in the CT scan is restricted or filtered to only (or mostly) show the patient. In some cases, the default CT scan also shows the table on which the patient is lying. In some cases, the table can be removed the image data with basic thresholding because the HU intensity values for a patient are considerably distinct from those of the inanimate table. In some cases, −80 HU effectively removes the table with negligible impact on patient data.

In some cases, the system can use Partial-Body CT Scans or Full-Body Scans. Full-body scans can provide reference points on the body to unambiguously resolve the patient's shape and pose and so infer an avatar. In some cases, the system can convert partial-body scans into synthesized full-body phantom templates personalized to patients. In some cases, to do so, for a given patient, a template can be selected from a library (e.g., the Extended Cardiac-Torso Phantom (XCAT) library) of phantom templates associated with different weight, height, or genders, etc. The one with the most similar weight, height, and gender can be selected as the patient (e.g., with height±1 inch of the patient and weight within ±10 kg). By default, the phantoms can provide attenuation coefficients these can be converted into HU units using Equation 1. The full-body template can be refined to better capture nuances of the patient's partial-body scan (e.g., body shape) by, for example, aligning the two data sources using the “Symmetric Normalization Diffeomorphic Registration with Mutual Information as the metric (SyN)”, which is featured in the open-source tool 3D Slicer. The result can be exported into the same standard medical imaging format as the original CT scan (e.g., Digital Imaging and Communications in Medicine, or DICOM).

In some cases, to create a Patient-Specific 3D Avatar with a Skeletal Anatomy, the system can convert the patient's CT scan into the personalized 3D avatar. In some cases, it can do so with three, sequential steps: infer a body mesh that in turn enables creating a body surface model that in turn is used for creating a skeleton model.

Body Mesh. Full-body scans, which can be a series of 2D slices, can be converted into a 3D surface mesh, for example using a “segment editor” feature in the open-source tool 3D Slicer. This method takes as input two threshold values specifying the lower and upper values to retain in the generated segmentation. As an example, these values can be set to 10 HU and 2000 HU respectively. In some cases, this feature segments all parts of the patient, from internal organs for inner values to the skin surface for outer values. In some cases, it only exports the segment's surface mesh and so a 3D skin-level representation.

Body Surface Model. A Skinned Multi Person Linear Model (SMPL) can be used to convert the full-body skin mesh into a 3D body surface model. A SMPL model is a statistical parametric function M(β, θ, t, Φ) whose output is a triangulated mesh with 6890 vertices (e.g., the number of vertices generally used for generating the skeleton model). Here β are the shape parameters, θ are the pose parameters, t is the global translation, and Φ are the learned model parameters—a distribution reflecting the body shapes of the general population. β defines how the body shape of a template body model must be changed to match the body shape of the original full-body skin mesh and θ define how the 24 joints defined in the SMPL model need to rotate relative to each other to match the pose of the original mesh.

Skeleton Model. A personalized skeleton model can be generated for a patient (e.g., each patient) from their SMPL body model, for example using an Obtaining Skeletal Shape from Outside (OSSO) model. The OSSO model is a parametric skeleton shape model learned from 2400 Dual X-Ray Absorptiometry Scans (DXA)—1200 male and 1200 female subjects—that can be represented as M(β, t, r), where β are the shape parameters, t are the translation parameters, and r are the rotation parameters for all the skeletal parts (29 parameters in total). The patient's pose can be matched. For example, the system can use the Stitched Puppet model, which allows each bone to move independently before stitching them together into a coherent skeleton. In some cases, the system uses pose-normalized skeleton models of the patients (e.g., in lying down position with hands in coronal plane.)

### Example Embodiments

Evaluation of at least some of the benefits of the disclosure inventive concepts can be achieved by: (1) comparing predicted skeletons to ground truth skeletons and (2) assessing the longitudinal performance of the predicted skeletons, where it is assumed a CT scan is only acquired on the first day of the treatment plan. Both can be evaluated with a common metric for comparing predicted skeleton meshes against the corresponding ground truth skeleton meshes: Root Mean Squared Error (RMSE). In this case, the ground truth was established using the TotalSegmentator feature in 3D Slicer that segments and exports all bones in ground truth CT scans as a single mesh.

The solution was evaluated on a retrospective dataset for 49 patients. It includes 198 CT scans with 1 to 11 scans per patient taken on different dates during their treatment, which spanned 8 weeks to 129 months. Scans were either full-body or partial-body from the skull to the femur. The dataset also includes the height(m), weight(lbs), gender, and age for most patients. For the 6 patients lacking height and weight data, values were imputed using the average height and weight for the adult U.S. population based on the patient's gender.

### Single CT Scan Assessment

The inventive concepts were evaluated for each of the 198 scans in the dataset.

Treatment-Relevant Bone Groups: Observing that radiation therapy is typically targeted to specific body regions, accuracy for specific bone groups is more clinically relevant than the accuracy of the total skeletal positioning. As a result, the AGRT was evaluated with respect to specific bone groups often used as landmarks during treatment: ribcage, vertebrae, scapula-clavicle, hips, and femur. An RMSE score per patient was calculated by taking the mean score across all the patient's CT scans for each bone group.

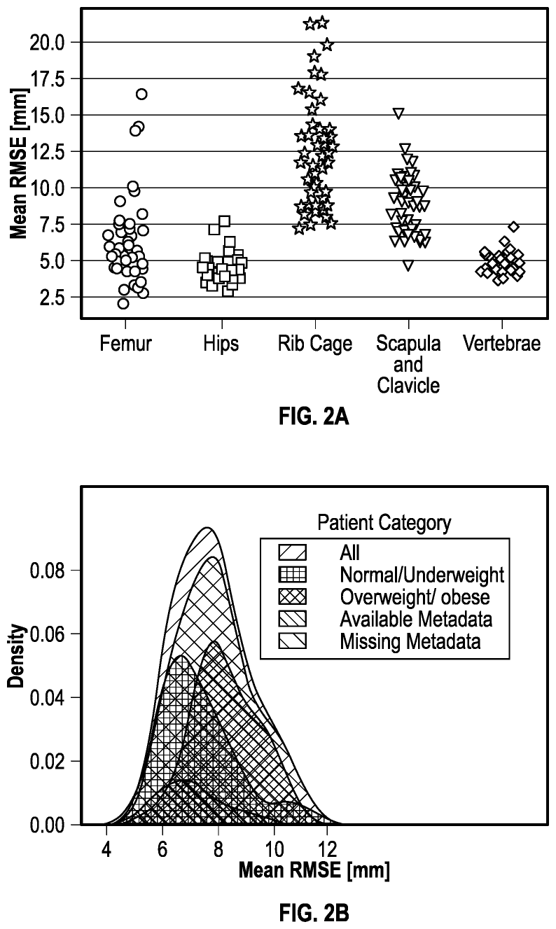

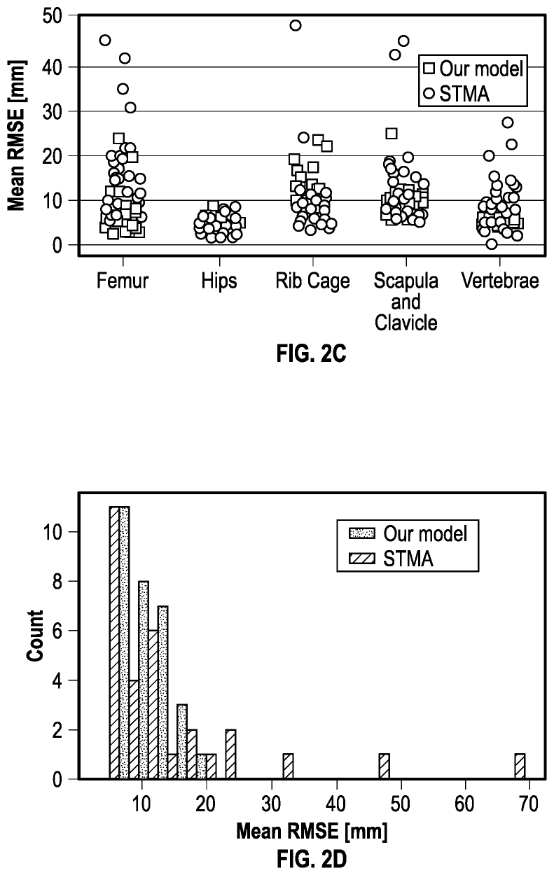

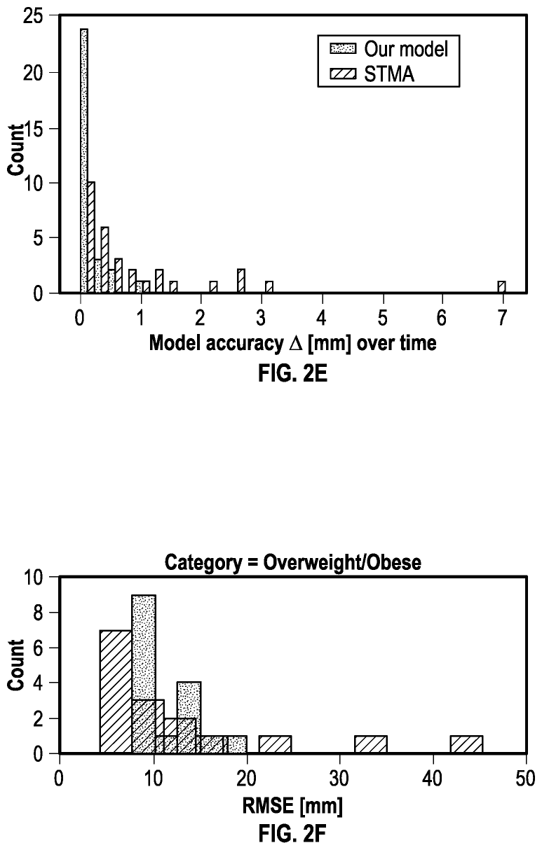

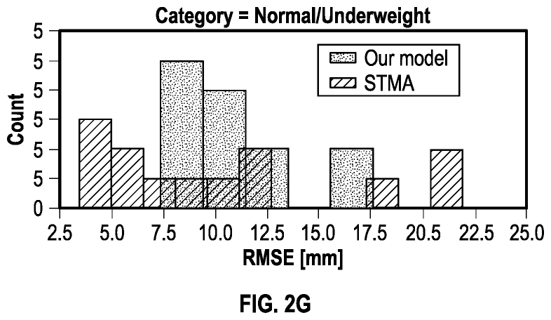

FIG. 2A illustrates example performance of an example Avatar Guided Radiation Therapy (AGRT) system, showing the Root Mean Square Error (RMSE) across various anatomical bone groups (Femur, Hips, Rib cage, Scapula and Clavicle, and Vertebrae). Each dot represents the RMSE for a specific bone group within a patient, separated by the bone group to indicate variance in accuracy and precision of the model across different anatomical features.

As shown, the hips and vertebrae exhibited the lowest variation in error (RMSE), while the ribcage, femur, scapula, and clavicle demonstrated greater variation. In some cases, the ribcage variation stems from patients' differing chest expansion or contraction on different scan dates. Respiratory motion is a known issue for accurate patient alignment in radiation therapy (RT), where motion of the thorax and abdomen may be on the order of centimeters. If this motion is not correctly managed, it can lead to under- or over-doses of radiation depending on the location of the target of radiation. Various techniques for motion management (MM) are used clinically. Incorporating a breathing motion model into the surface model has overcome these increased errors and allowed the technique to accurately model breathing motion. Conversely, pose variations between scans might explain the higher variability observed in the femur, scapula, and clavicle. Mapping the patient's real-time movements to the avatar resolved the errors due to pose variations.

Overall Skeleton: The overall similarity between the predicted and ground truth skeletons was evaluated. In this evaluation, only mesh points on the predicted skeletons that are available in the ground truth skeleton meshes were considered.

FIG. 2B shows a density plot of the mean RMSE values for overall skeletal accuracy, categorized by patient demographics, to demonstrate example adaptability and effectiveness of the AGRT system across different patient conditions.

As shown, the mean RMSE of all a patient's scans, for 91.84% of the patients, is less than 10 mm. While sub-centimeter positioning may not be precise enough for image-guided RT, initial patient positioning to within a centimeter has reduced the frequency of X-Ray based image guidance, and thus the additional imaging dose.

FIGS. 2C-2G illustrates a comparison of an example AGRT solution and the status quo in clinical settings of STMA with respect to (2C) treatment-relevant bone groups, (2D, 2E) overall skeletons, and (2F, 2G) influence of body mass index.

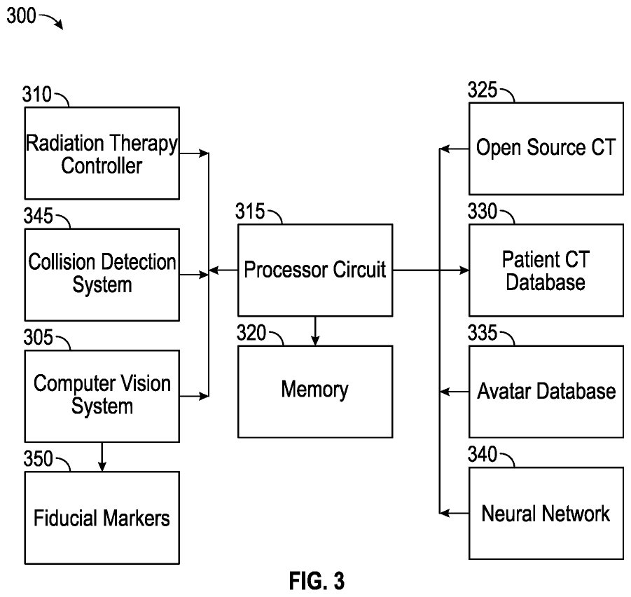

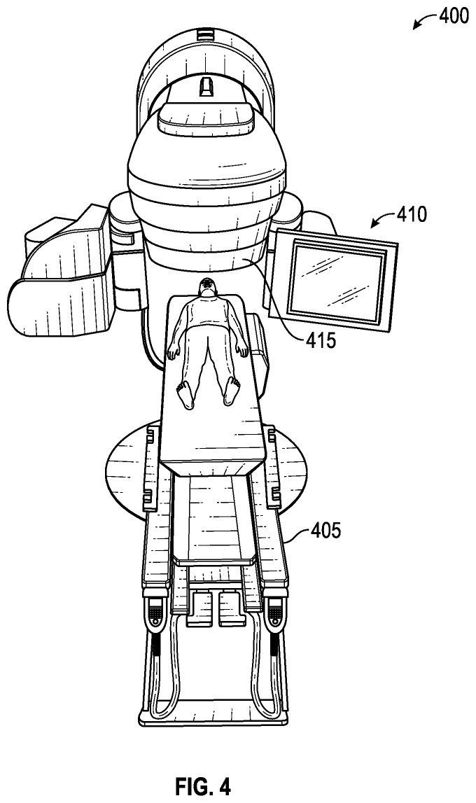

FIG. 3 is a block diagram illustrating an example SGRT system 300, in accordance with some embodiments of the inventive concepts. The SGRT system 300 includes a computer vision system 305 configured to capture the pose of a patient in the SGRT environment to generate a patient specific skin mesh model and including a real-time therapy guidance controller 310 to control targeting, dosage, and/or timing of the radiation delivered during therapy. The real-time therapy guidance controller 310 can be configured to compensate for the movement of the patient's anatomy based on the patient's breathing in some embodiments according to the present inventive concept.

According to FIG. 3, the system 300 can include a processor circuit 315 coupled to a memory 320 that can be configured to store instructions for operations of the processor circuit 315 and data, such as models trained by the system 300 (or other systems). The models stored in the memory 320 can include the various models and related data described herein. It will be understood that the models may also be stored in one or more databases that can be operatively coupled to the processor circuit 315 and that can be loaded into the memory 320 for use by the processor circuit 315 to carry out the operations described herein.

The system 300 also includes access to an avatar database 335 that stores models of human body shapes (referred to as “avatars”). An avatar that represents a good fit for the patient body type can be selected for development of a patient specific skin mesh model that will be used to administer the therapy to the specific patient. The avatar selected for the patient can be modified based on, for example, but not limited to, the pose of the patient in the environment, the gender of the patient, and/or the BMI of the patient to provide the patient specific skin mesh model. In some embodiments according to some inventive concepts, the avatars can be generated from a database of 3D scans of a large number of human bodies. In some embodiments according to some inventive concepts, the avatars can be based on the Sparse Trained Articulated Human Body Regressor (STAR) published in Osman A A A, Bolkart T, Black M J. STAR: Sparse Trained Articulated Human Body Regressor. In: Vol 32351.; 2020:598-613. doi:10.1007/978-3-030-58539-6_36.

The system 300 can include access to a neural network 340 that is trained to provide a prediction of the location of a skeleton for an oncology patient derived from the patient specific skin mesh model. The patient skeleton location can be used to determine a predicted location of a particular part of the patient anatomy/tumor in the particular pose that the patient presents (given that the patient specific skin mesh model integrates the patient pose). In some embodiments according to some inventive concepts, the neural network 340 can be provided using the approach described in Keller M, Zuffi S, Black M J, Pujades S. OSSO: Obtaining Skeletal Shape From Outside. In: 2022:20492-20501.

The system 300 can include access to a patient CT database 330 that stores CT data for each specific patient. The CT data for a specific patient can be used to align the predicted location of the particular part of the patient anatomy/tumor, provided by use of the neural network 340, to the patient's anatomy when the patient is positioned in the environment for the therapy. In some embodiments according to some inventive concepts, an iterative closest point algorithm can be used to rigidly register to the skeletal anatomy such that the distance between them is minimized.

The system 300 can include a collision detection system 345 that is configured to compare the position of the patient in the environment with the path of a gantry, including the radiation source etc., during movement. The position of the patient and the path of the gantry can be determined using the computer vision system 305. The collision detection system 345 can interrupt or disable movement of the gantry if the position of the patient and the path of the gantry are determined to occupy the same location in the environment during a planned therapy. Accordingly, the system 300 and the gantry and/or radiation source may not need to be integrated into a single system and, moreover, the system 300 can provide the collision detection with a wide range of gantries and radiation sources from various system providers.

Still referring to FIG. 3, fiducial markers 350 can be placed in the patient during therapy. The computer vision system 305 can capture images of the patient in the environment and determine the location and movements of the fiducial markers 350 as the patient breathes. It will be understood that the system 300 can optionally include a monitor coupled to the patient to provide an indication of the patient's breathing cycle to the system 300 during therapy. In some embodiments, the computer vision system 305 can track the fiducial markers 350 alone to monitor the patient's breathing cycle.

During therapy, the motion of the patient's skin due to breathing (indicated by the movement of the fiducial markers 350) can be used to estimate the related movement of the patient's anatomy. In some embodiments according to the present inventive concept, the movement of the patient's anatomy caused by breathing can be determined as described in Tsoli A, Mahmood N, Black M J. Breathing life into shape: capturing, modeling and animating 3D human breathing. ACM Trans Graph. 2014; 33(4):52:1-52:11. doi:10.1145/2601097.2601225. In some embodiments according to the present inventive concept, other methods can be used.

In some embodiments according to the present inventive concept, an image registration system can be used to determine the motion from the pre-treatment 4D-CT which can be and fitted to a correspondence model relating the motion due to breathing to the skin surface. Using the technique outlined in Guo H, Planche B, Zheng M, Karanam S, Chen T, Wu Z. SMPL-A: Modeling Person-Specific Deformable Anatomy. In: 2022:20814-20823. Accessed Aug. 30, 2022. https://openaccess.thecvf.com/content/CVPR2022/html/Guo_SMPL-A_Modeling_Person-Specific_Deformable_Anatomy_CVPR_2022_paper.html, a pose-dependent organ deformation can be applied, generated using a point cloud autoencoder conditioned on the parametric pose input, to deform the skin mesh model and internal anatomy to the individual breathing phases observed during treatment.

Still referring to FIG. 3, the processor circuit 315 can operate to coordinate overall operation of the components included in the system 300 to create the models prior to therapy and use models to carry out therapy in real time. It will be understood that although the system 300 is shown as having the illustrated components integrated into a single system, the operations and components described herein can be included in and performed by different systems that can be separate from one another.

It will also be understood that the term “real-time” includes various operations in computing or controlled processes that provide response times within a specified time. It will be understood that the term “real-time” process is generally one that happens in defined time steps of maximum duration and with a latency that is little enough so that the process can affect the environment in which it occurs, such as adjusting radiation therapy time and location responsive to movement or tumors based on, for example, patient breathing. It will be understood that although embodiments according to the present inventive concept are described with reference to SGRT, the scope of the present inventive concept includes other type of radiation therapy used in oncology.

FIG. 4 is a schematic illustration of a patient in a particular pose on a platform 405 in the SGRT environment 400 in some embodiments according to the present inventive concept. The system 300, described in FIG. 3, is located proximate to the environment 400 so that the computer vision system 305 can determine the pose of the patient. The patient is positioned near a gantry 410 that is coupled to a radiation source 415 that provides the therapy to the patient under control of the system 300. The gantry 410 and the radiation source 415 can move relative to the patient and the platform 405 during the therapy. The collision avoidance system 345 can monitor the environment 400 in real-time to prevent the gantry 410 (and all components included therewith) from colliding with the patient during the therapy.

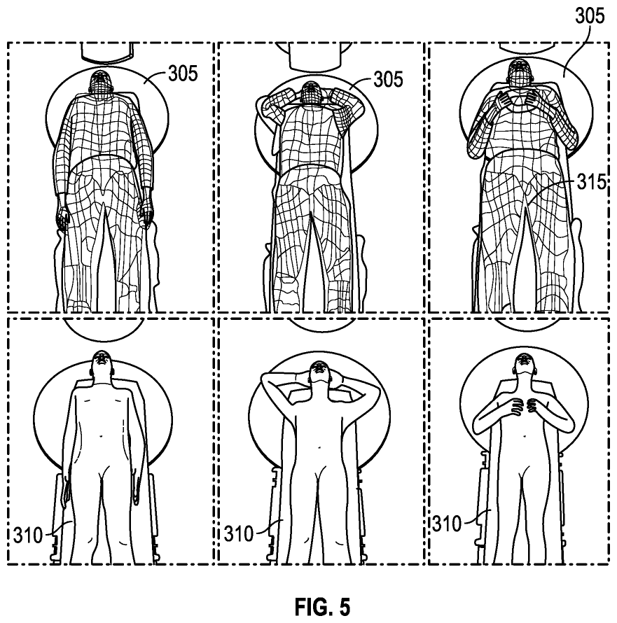

FIG. 5 is a schematic illustration of three exemplary patients 505 in respective poses on the platform captured by the computer vision system 305 and the associated avatar 510 selected for each patient 505 in the pose based on a plurality of patient characteristics in some embodiments according to the present inventive concept. As shown in FIG. 5, each of the avatars 510 reflects the respective body type of the patient 505 that can affect the location of the patient anatomy relative to the surface of the patient skin. Moreover, the avatars 510 can be modified to reflect the patient pose as well as other patient characteristics. During operation, the computer vison system 305 captures the pose of the patient 505 and the selected avatar 510 can be modified to reflect the pose and other characteristics of the patient 505 to generate the patient specific skin mesh model 515 shown projected onto the respective patient 505.

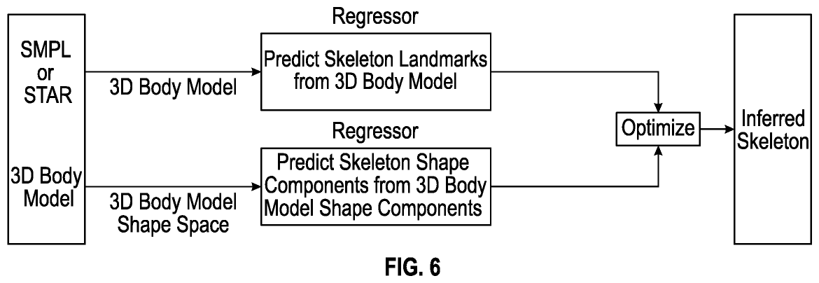

FIG. 6 is a block diagram of processing operations configured to generate a patient specific skeletal model based on the patient specific skin mesh model 515, as described in reference to FIGS. 3 and 5, wherein the skeletal model is modified using patient specific CT data to align the skeletal model to the patient skeleton to provide the patient specific skeletal position model in some embodiments according to the present inventive concept. As described in reference to FIG. 3, in some embodiments according to the present inventive concept, the patient specific skeletal position model can be derived from the patient specific skin mesh model as described in, for example, Keller M, Zuffi S, Black M J, Pujades S. OSSO: Obtaining Skeletal Shape From Outside. In: 2022:20492-20501.

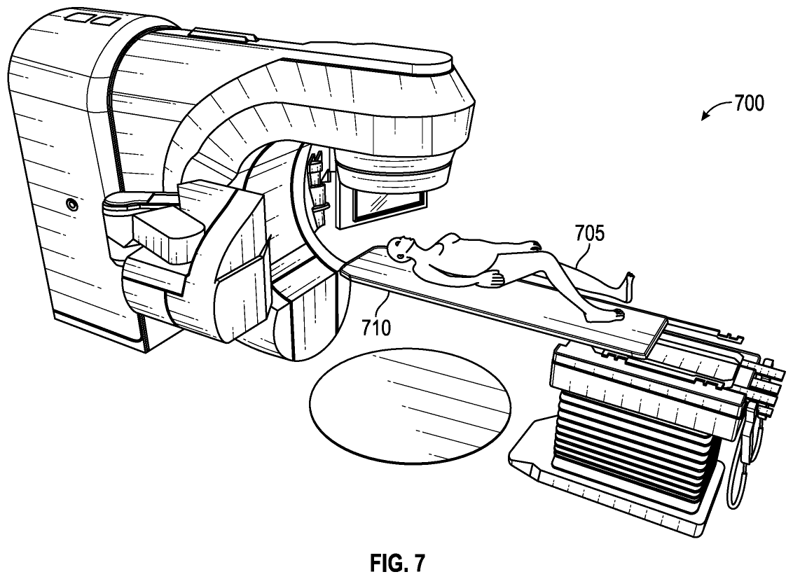

FIG. 7 is an illustration of selected avatar 705 in a particular pose on a platform 710 in the SGRT environment 700, the selected avatar 705 is selected based on a body type of the patient in some embodiments according to the present inventive concept.

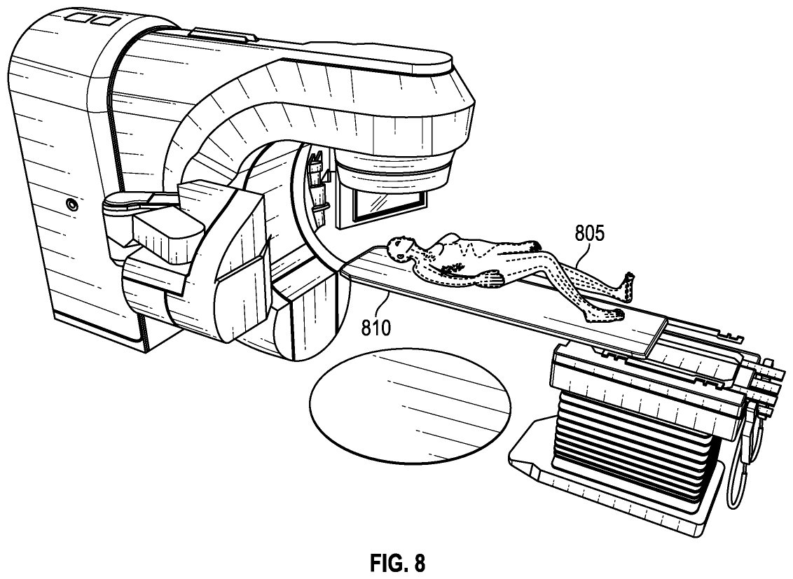

FIG. 8 is an illustration of the selected patient avatar 705 in FIG. 7 having been modified based on patient pose, gender, race, and BMI to provide a patient avatar 805 annotated with a patient specific skin mesh model 810 thereon in some embodiments according to the present inventive concept.

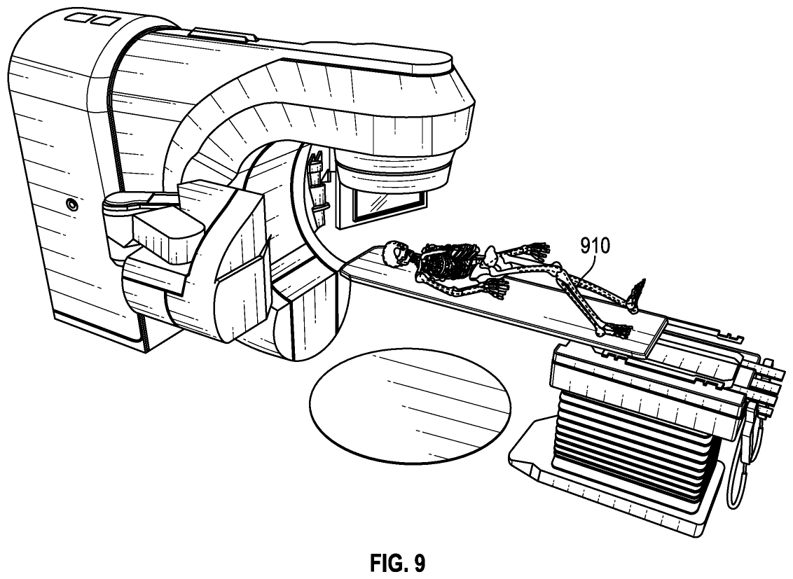

FIG. 9 is an illustration of the patient avatar 805 in FIG. 8 annotated with a patient specific skeletal position model 910 generated based on the patient specific skin mesh model as shown in FIG. 8 and FIG. 3 in some embodiments according to the present inventive concept. As described in reference to FIG. 3, the system 300 includes access to the neural network 340 that is trained to generate the patient specific skeletal position model to provide a prediction of the location of a patient skeleton 910 from the patient specific skin mesh model 810. The patient specific skeletal position model can be used to determine a predicted location of a particular part of the patient anatomy/tumor when in the particular pose that the patient presents (given that the patient specific skin mesh model integrates the patient pose). In some embodiments according to some inventive concepts, the neural network 340 can be provided using the approach described in Keller M, Zuffi S, Black M J, Pujades S. OSSO: Obtaining Skeletal Shape From Outside. In: 2022:20492-20501.

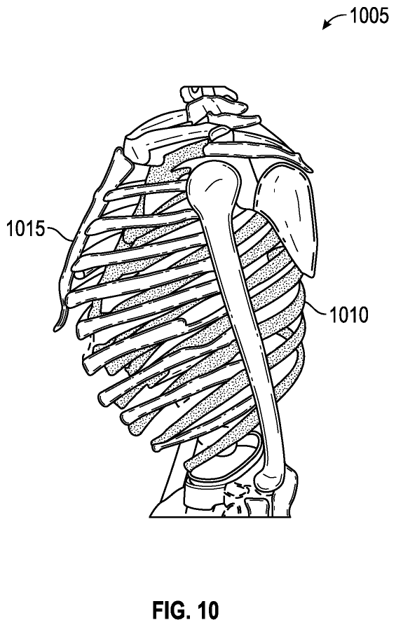

FIG. 10 is an illustration of a specific patient 1005 annotated to reflect patient skin movement due to breathing which is used to determine anatomical movement in some embodiments according to the present inventive concept. As described in reference to FIG. 3, the fiducial markers 350 placed can be placed on the patient 1005 during therapy. The computer vision system 305 can capture images of the patient 1005 in the environment and determine the location of the patient skin surface at different times during the breathing cycle using the fiducial markers 350. As shown in FIG. 10, as the patient 1005 inhales, the skeletal structure is determined to move from position 1010 to position 1015. It will be understood that the system 100 can optionally include a monitor coupled to the patient 1005 to provide an indication of the patient's breathing cycle to the system 100 during therapy. In some embodiments, the computer vision system 305 can track the fiducial markers 350 alone to monitor the patient's breathing cycle.

During therapy, the motion of the patient's skin due to breathing (indicated by the movement of the skeletal structure) can be used to estimate the related movement 1005 of the patient's anatomy. In some embodiments according to the present inventive concept, the movement of the patient's anatomy 1005 caused by breathing can be determined as described in Tsoli A, Mahmood N, Black M J. Breathing life into shape: capturing, modeling and animating 5D human breathing. ACM Trans Graph. 2014; 33(4):52:1-52:11. doi:10.1145/2601097.2601225. In some embodiments according to the present inventive concept, other methods can be used.

In some embodiments according to the present inventive concept, an image registration system can be used to determine the motion from the pre-treatment 6D-CT which can be and fitted to a correspondence model relating the motion due to breathing to the skin surface. Using the technique outlined in Guo H, Planche B, Zheng M, Karanam S, Chen T, Wu Z. SMPL-A: Modeling Person-Specific Deformable Anatomy. In: 2022:20814-20823. Accessed Aug. 30, 2022. https://openaccess.thecvf.com/content/CVPR2022/html/Guo_SMPL-A_Modeling_Person-Specific_Deformable_Anatomy_CVPR_2022_paper.html, a pose-dependent organ deformation can be applied, generated using a point cloud autoencoder conditioned on the parametric pose input, to deform the skin mesh model and internal anatomy to the individual breathing phases observed during treatment.

### Flow Diagram

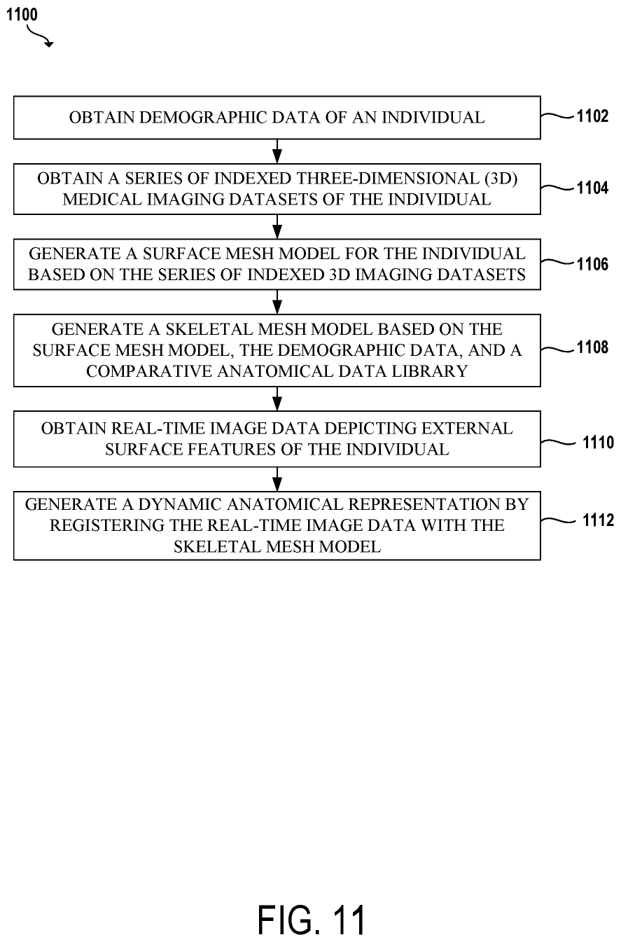

FIG. 11 is a flow diagram illustrative of an embodiment of a routine 1100 for generating a dynamic anatomical representation of an individual, suitable for assisting in guiding adaptive procedures. Although described as being implemented by the processor circuit 315, it will be understood that one or more elements outlined for routine 1100 can be implemented by one or more computing devices/components that are associated with an SGRT system, such as system 300. Thus, the following illustrative embodiment should not be construed as limiting.

At block 1102, the processor circuit 315 can obtain demographic data of an individual. This demographic data can include an indication of at least one characteristic, such as gender, race, or body mass index (BMI). The data can be acquired from various sources, including electronic health records, patient-reported information, or healthcare databases. The demographic data can be stored in a structured format, enabling efficient comparison and analysis during subsequent steps of the adaptive procedure. The processor circuit 315 can use the demographic data to customize and refine the selection of anatomical templates, ensuring that comparative analysis aligns with the characteristics and unique profile of the individual.

At block 1102, the processor circuit 315 can obtain demographic data of an individual. The demographic data can include an indication of at least one characteristic, such as gender, race, body mass index (BMI), age, ethnicity, height, weight, genetic predispositions, smoking status, existing comorbidities, or the like. The demographic data can be obtained from various sources, including, but not limited to, electronic health records, patient questionnaires, wearable health devices, or healthcare databases. In some cases, the demographic data can be stored in a structured format, enabling efficient comparison, analysis, or retrieval.

At block 1104, the processor circuit 315 can obtain a series of indexed three-dimensional (3D) medical imaging datasets of the individual. In some cases, each dataset can correspond to a distinct phase of the respiratory cycle of the individual, depicting internal anatomical structures such as the lungs, diaphragm, and/or surrounding organs. The series can include at least two medical imaging datasets. As a nonlimiting example, one dataset can correspond to an inhalation phase (e.g., full inhalation, mid-inhalation, etc.) and one dataset can correspond to an exhalation phase (e.g., full exhalation, mid-exhalation, etc.) of the individual's respiratory cycle. In addition or alternatively, the series can, in some cases, include additional dataset, such as those corresponding to additional phases, such as full inhalation, mid-inhalation, full exhalation, mid-exhalation, and/or other intermediate stages.

In some cases, the series of indexed 3D computed tomography (CT) datasets collectively form a four-dimensional (4D) CT dataset, thereby capturing dynamic anatomical changes over time due to the individual's respiratory cycle. The processor circuit 315 can organize these datasets to ensure consistent alignment and comparison between phases, providing spatial and temporal information about internal movement patterns and anatomical changes for accurate dynamic anatomical representation.

In some cases, the individual has a target region, such as, but not limited to, a tumor, metastatic lesions, arteriovenous malformations (AVMs), benign growths, proliferative vascular lesions, or hyperfunctioning endocrine tumors. The series of indexed three-dimensional (3D) medical imaging datasets can include information pertaining to the target region, such as its location, size, and/or shape. Additionally or alternatively, the processor circuit 315 can obtain target region data separately, with the target region data comprising an indication of the location, size, and/or shape of the target region. The generation of the dynamic anatomical representation can include incorporating this target region data into the skeletal mesh model to provide a representation specifically focused on the target region. By integrating this data into the model, the processor circuit 315 can facilitate accurate identification, monitoring, and tracking of the target region throughout different phases of the individual's respiratory cycle.

At block 1106, the processor circuit 315 can generate a surface mesh model for the individual based on the series of indexed three-dimensional (3D) imaging datasets. The surface mesh model can represent an external contour of the body of the individual. In some cases, the surface mesh model can be the patient specific skin mesh model described herein. This process of generating the surface mesh model can include, but is not limited to, aligning and processing the 3D imaging datasets to capture accurate external contours. The surface mesh model can reflect variations in external anatomical features across the respiratory cycle, enabling a representation of the individual's body contours for adaptive procedure planning and guidance.

At block 1108, the processor circuit 315 can derive a skeletal mesh model using the surface mesh model, demographic data, and/or a comparative anatomical data library. The surface mesh model can provide detailed information about the external contour of the individual's body, offering a reference for the processor circuit 315. The demographic data can include parameters such as age, gender, race, height, weight, and body mass index (BMI), which can guide the processor circuit in customizing the skeletal mesh model. The comparative anatomical data library can include a diverse set of anatomical templates, each containing internal skeletal configurations representing different body types and demographic variations.

The processor circuit 315 can select an appropriate anatomical template from the data library by comparing the individual's demographic data with predefined thresholds for anatomical congruence. These thresholds can include criteria such as differences in height, body mass index (BMI), or age between the template and the individual. For instance, the comparison can identify deviations in height within a range of 5 cm, BMI differences up to 2 points, or age differences of up to 5 years. This allows the selected template to closely approximate the individual's expected internal skeletal structure. The processor circuit 315 can then refine this template using the surface mesh model to establish accurate correlations between the external surface features and the internal skeletal configurations, resulting in a detailed skeletal mesh model that provides an individualized skeletal representation.

At block 1110, the processor circuit 315 can obtain real-time image data depicting the external surface features of the individual. The real-time image data can be collected using a computer vision system, such as computer vision system 305, which may include cameras and/or optical sensors strategically placed to capture different angles of the individual's body. The computer vision system can be configured to detect fiducial markers placed on the individual's skin or directly detect anatomical features, such as contours of limbs and facial features. This data can be processed to provide a continuous stream of movement patterns and postural changes, enabling real-time tracking of respiratory cycles, skeletal shifts, and/or other relevant anatomical movements. The real-time image data can provide valuable insight into how external features reflect internal skeletal positioning.

At block 1112, the processor circuit 315 can generate a dynamic anatomical representation by registering the real-time image data with the skeletal mesh model. The registration process can include aligning the external features captured in real-time with the corresponding internal skeletal configurations to create an accurate, dynamic representation. This dynamic anatomical representation can adapt to reflect changes in the individual's pose, posture, or movement over time, as indicated by shifts in the real-time image data. Such adaptation can account for various anatomical changes during different phases of the respiratory cycle, ensuring that the dynamic anatomical representation remains updated and closely aligned with the individual's current state.

In some cases, a dynamic anatomical representation can be used to assist healthcare professionals in planning or simulation purposes, providing anatomical insights that can help refine a radiation therapy plan. By continuously updating to reflect real-time changes in pose and posture, the dynamic anatomical representation offers a reliable framework for predicting how internal structures may shift over different phases of the respiratory cycle. This information can guide adjustments to radiation therapy plans, ensuring accurate and consistent targeting of the treatment area. In some cases, the dynamic anatomical representation can directly inform the delivery of radiation therapy by controlling dosage and timing. Radiation can be activated when the target region aligns within the predetermined focal area of a therapeutic beam and deactivated when the region moves out of alignment. This adaptive approach minimizes unnecessary radiation exposure to surrounding healthy tissues, ultimately improving treatment outcomes.

### Terminology

Any or all of the features and functions described above can be combined with each other, except to the extent it may be otherwise stated above or to the extent that any such embodiments may be incompatible by virtue of their function or structure, as will be apparent to persons of ordinary skill in the art. Unless contrary to physical possibility, it is envisioned that the methods/operations described herein may be performed in any sequence and/or in any combination, and the components of respective embodiments may be combined in any manner.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims, and other equivalent features and acts are intended to be within the scope of the claims.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or operations. Thus, such conditional language is not generally intended to imply that features, elements and/or operations are in any way required for one or more embodiments or that one or more embodiments necessarily include logic for deciding, with or without user input or prompting, whether these features, elements and/or operations are included or are to be performed in any particular embodiment.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number, respectively. The word “or” in reference to a list of two or more items, covers all of the following interpretations of the word: any one of the items in the list, all of the items in the list, and any combination of the items in the list. Likewise, the term “and/or” in reference to a list of two or more items, covers all of the following interpretations of the word: any one of the items in the list, all of the items in the list, and any combination of the items in the list.

Conjunctive language such as the phrase “at least one of X, Y and Z,” unless specifically stated otherwise, is otherwise understood with the context as used in general to convey that an item, term, etc. may be either X, Y or Z, or any combination thereof. Thus, such conjunctive language is not generally intended to imply that certain embodiments require at least one of X, at least one of Y and at least one of Z to each be present. Further, use of the phrase “at least one of X, Y or Z” as used in general is to convey that an item, term, etc. may be either X, Y or Z, or any combination thereof.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

Any patents and applications and other references noted above, including any that may be listed in accompanying filing papers, are incorporated herein by reference. Aspects of some inventive concepts can be modified, if necessary, to employ the systems, functions, and concepts of the various references described above to provide yet further implementations of some inventive concepts. These and other changes can be made to some inventive concepts in light of the above Detailed Description. While the above description describes certain examples of some inventive concepts, and describes the best mode contemplated, no matter how detailed the above appears in text, some inventive concepts can be practiced in many ways. Details of the system may vary considerably in its specific implementation, while still being encompassed by some inventive concepts disclosed herein. As noted above, particular terminology used when describing certain features or aspects of some inventive concepts should not be taken to imply that the terminology is being redefined herein to be restricted to any specific characteristics, features, or aspects of some inventive concepts with which that terminology is associated. In general, the terms used in the following claims should not be construed to limit some inventive concepts to the specific examples disclosed in the specification, unless the above Detailed Description section explicitly defines such terms. Accordingly, the actual scope of some inventive concepts encompasses not only the disclosed examples, but also all equivalent ways of practicing or implementing some inventive concepts under the claims.

To reduce the number of claims, certain aspects of some inventive concepts are presented below in certain claim forms, but the applicant contemplates other aspects of some inventive concepts in any number of claim forms. Any claims intended to be treated under 35 U.S.C. § 112(f) will begin with the words “means for,” but use of the term “for” in any other context is not intended to invoke treatment under 35 U.S.C. § 112(f). Accordingly, the applicant reserves the right to pursue additional claims after filing this application, in either this application or in a continuing application.

1. A method for generating a dynamic anatomical representation of an individual, suitable for assisting in guiding adaptive procedures, comprising:
obtaining demographic data of an individual, the demographic data comprising an indication of at least one of gender, race, or body mass index (BMI);
obtaining a series of indexed three-dimensional (3D) medical imaging datasets of the individual, each dataset corresponding to a distinct phase of a respiratory cycle of the individual and depicting internal anatomical structures of the individual;
generating a surface mesh model for the individual based on the series of indexed 3D imaging datasets, wherein the surface mesh model represents an external contour of a body of the individual;
deriving a skeletal mesh model based on the surface mesh model, the demographic data, and a comparative anatomical data library, wherein the comparative anatomical data library includes a set of anatomical templates, and wherein deriving the skeletal mesh model comprises:
using the demographic data to select an anatomical template that satisfies predefined thresholds for anatomical congruence with characteristics of the individual as represented by the demographic data, and
correlating external surface features from the surface mesh model with internal skeletal configurations of the selected anatomical template;
obtaining real-time image data depicting external surface features of the individual; and
generating a dynamic anatomical representation by registering the real-time image data with the skeletal mesh model, wherein the dynamic anatomical representation adapts to reflect changes in a pose of the individual.

2. The method of claim 1, wherein the series of indexed three-dimensional (3D) computed tomography (CT) imaging datasets includes at least two datasets, one corresponding to a full inhalation phase and one corresponding to a full exhalation phase of a respiratory cycle of the individual.

3. The method of claim 1, wherein each of the series of indexed three-dimensional (3D) imaging datasets is a computed tomography (CT) dataset.

4. The method of claim 1, wherein the series of indexed three-dimensional (3D) computed tomography (CT) datasets collectively form a four-dimensional (4D) CT dataset, capturing dynamic anatomical changes over time due to the respiratory cycle of the individual.

5. The method of claim 1, further comprising updating the surface mesh model and the skeletal mesh model continuously using a computer vision system to reflect real-time movements of the individual, wherein updates include adjustments based on detected breathing cycles of the individual.

6. The method of claim 1, wherein obtaining real-time image data comprises capturing images of fiducial markers placed on an external surface of the individual, wherein the captured images are used to track movement due to respiratory and other body movements.

7. The method of claim 6, wherein motion of skin of the individual, as indicated by the movement of the fiducial markers, is utilized to estimate related movements of an anatomy of the individual.

8. The method of claim 1, wherein the dynamic anatomical representation includes a predictive model of skeletal movement derived from the skeletal mesh model and the real-time image data, wherein the predictive model accounts for variations in skeletal position due to respiratory movements.

9. The method of claim 1, wherein the demographic data is used to refine the anatomical template selection by comparing a height, weight, and/or gender of the individual with templates from the comparative anatomical data library to find a closest match.

10. The method of claim 1, wherein radiation therapy is applied using the dynamic anatomical representation to control dosage and/or timing of radiation.

11. The method of claim 1, further comprising obtaining target region data, the target region data comprising an indication of a location, size, and/or shape of a target region, wherein generating the dynamic anatomical representation includes incorporating the target region data into the skeletal mesh model to provide a target region-specific representation.

12. The method of claim 11, wherein the dynamic anatomical representation is configured to track changes in target region location relative to the skeletal mesh model across distinct respiratory phases to provide adaptive guidance.

13. The method of claim 12, further comprising predicting potential movements of the target region based on respiratory phases of the individual, wherein the skeletal mesh model is updated to reflect anticipated target region shifts during the adaptive guidance.

14. The method of claim 11, further comprising delivering radiation therapy by activating a therapeutic beam when the target region aligns within a predetermined focal area, as indicated by the dynamic anatomical representation.

15. The method of claim 1, wherein the dynamic anatomical representation is intended for planning or simulation purposes, to inform healthcare professionals in developing or refining a radiation therapy plan.

16. A system for generating a dynamic anatomical representation of an individual, the system comprising:
a processor circuit, operatively coupled to a computer vision system, wherein the processor circuit is configured to:
obtain a series of indexed three-dimensional (3D) medical imaging datasets of the individual, each dataset corresponding to a distinct phase of a respiratory cycle of the individual and depicting internal anatomical structures of the individual;
generate a surface mesh model based on the series of indexed 3D imaging datasets, wherein the surface mesh model represents an external contour of the body of the individual;
derive a skeletal mesh model from the surface mesh model using demographic data of the individual and a comparative anatomical data library, wherein the comparative anatomical data library includes a set of anatomical templates, and wherein deriving the skeletal mesh model comprises:
using the demographic data to select an anatomical template that satisfies predefined thresholds for anatomical congruence with characteristics of the individual, and
correlating external surface features from the surface mesh model with internal skeletal configurations of the selected anatomical template;
obtain real-time image data depicting external surface features of the individual; and
register the real-time image data with the skeletal mesh model to generate a dynamic anatomical representation that adapts to reflect changes in the pose of the individual.

17. The system of claim 16, wherein the series of indexed three-dimensional (3D) computed tomography (CT) imaging datasets includes at least two datasets, one corresponding to a full inhalation phase and one corresponding to a full exhalation phase of a respiratory cycle of the individual.

18. The system of claim 16, wherein the processor circuit obtain tumor data of the individual, the tumor data comprising an indication of the location, size, and/or shape of a tumor, wherein the processor circuit generates the dynamic anatomical representation by incorporating the tumor data into the skeletal mesh model to provide a tumor-specific representation.

19. A computer-implemented method for adapting radiation therapy based on positioning of a target region, the method comprising:
obtaining a series of three-dimensional (3D) medical imaging datasets depicting an individual and a target region at different phases of a respiratory cycle of the individual;
generating a dynamic anatomical model of the individual based on the 3D imaging datasets, wherein the dynamic anatomical model includes a representation of the target region, wherein generating comprises:
constructing a surface mesh model representing an external contour of a body of the individual,
deriving an internal skeletal model from the surface mesh model by correlating external surface features with internal skeletal configurations using a comparative anatomical data library, and
registering the internal skeletal model with the representation of the target region from the 3D imaging datasets to create the dynamic anatomical model;
based on the dynamic anatomical model, determining an alignment of the target region with a predetermined focal area of a therapeutic beam during different phases of the respiratory cycle; and
controlling delivery of radiation therapy based on the alignment, wherein radiation is activated when the target region is within the predetermined focal area and deactivated when the target region moves out of the predetermined focal area.

20. The computer-implemented method of claim 19, wherein determining the alignment of the target region with the predetermined focal area is intended for planning or simulation purposes to inform the development or refinement of a radiation therapy plan.
